(19) United States
(12) Patent Application Publication
(10) Pub. No.: US 2006/0175209 A1
(43) Pub. Date: Aug. 10, 2006

(54) ORTHODONTIC APPLIANCE ORGANIZER

(75) Inventors: Jefferson Sabilla, Ontario, CA (US); Kevin Corcoran, Corona, CA (US)

Correspondence Address: WOOD, HERRON & EVANS, LLP, 2700 CAREW TOWER, 441 VINE STREET, CINCINNATI, OH, 45202, US

(73) Assignee: Ormco Corporation, Orange, CA (US)

(21) Appl. No.: 11/279,140

(22) Filed: Apr. 10, 2006

Related U.S. Application Data
(63) Continuation-in-part of application No. 10/781,138, filed on Feb. 18, 2004.

(51) Int. Cl. A61B 19/02

(52) U.S. Cl. 206/63.5

(57) ABSTRACT

An orthodontic appliance organizer for organizing and loading orthodontic appliances, for example orthodontic brackets, onto set-up trays. The orthodontic appliance organizer includes an organizer tray for holding a removable set-up tray support member and a corresponding set-up tray in a fixed orientation. The orthodontic appliance organizer also includes a storage component having storage compartments for storing the orthodontic appliances. The storage component is accessible by the user to obtain orthodontic appliances for placing into the organizer tray without moving the organizer tray or the storage component. Additional access to the storage component can be made by rotating a portion of the organizer tray to reload orthodontic appliances. The storage component, organizer tray, set-up tray support member and the orthodontic appliance organizer are stackable upon themselves.

### CROSS REFERENCE TO RELATED APPLICATIONS

This application is a continuation-in-part of prior U.S. application Ser. No. 10/781,138, filed on Feb. 18, 2004, the disclosure of which is hereby incorporated by reference in its entirety.

### TECHNICAL FIELD OF THE INVENTION

This invention relates to the packaging of dental appliances, particularly orthodontic appliances, and to the organizing of the appliances for their loading onto set-up trays. The invention particularly relates to methods of packaging and organizing of the appliances and of loading set-up trays with the appliances, and to the packages, organizers and set-up trays and appliance packaging, loading and organizing systems.

### BACKGROUND OF THE INVENTION

In the practice of dentistry, the efficient use of a dentist's time is enhanced by preparing the equipment and supplies needed by the dentist for a scheduled patient visit in advance of appointment, so that the dentist may proceed directly with an examination or treatment. The preparation is typically done by a technician or assistant on the dentist's staff. As a result, the dentist may see more patients in a specified period of time than would be possible if the dentist were to personally assemble the items.

Orthodontic practitioners, for example, have a number of examination or treatment activities, each of which may require different tools or supplies. A common task for an orthodontist is the installation of an orthodontic appliance on a patient. Currently, a most effective and widely used orthodontic appliance is an orthodontic brace that is formed of an archwire and a number of orthodontic brackets. Installation of such an appliance typically involves the bonding of the individual brackets of a set onto corresponding teeth of the patient. For such an installation, a set of brackets must be assembled for the orthodontist, along with adhesives, primers and other chemical substances, plus tweezers, curing light guns and other tools needed for the appliance installation.

The installation of a bracket-archwire appliance requires careful selection and placement of individual brackets from a supply onto the teeth usually in a one-by-one transfer of the brackets from the supply to the patient. The transfer involves the engagement of the correct bracket for a given tooth and the proper orientation of the bracket for placement on the appropriate tooth. Since the brackets are each tooth-specific, with each having a geometry specially configured to fit the surface of a tooth and to engage an archwire to exert specific forces on the tooth, an error in the selection of a bracket can be costly in terms of treatment time and loss of treatment quality.

Placement of a bracket on a tooth requires the orthodontist to hold the bracket, usually with a pair of tweezers designed for that purpose, with the base of the bracket coated with adhesive and oriented for placement against the patient's tooth. Each bracket base may be considered as having four sides, one intended as the gingival side that must face the gum when mounted on the tooth and one intended as the opposite occlusal side which will face the occlusal plane. At right angles to these are the mesial and distal sides, which will face the mesial and distal sides of the tooth, respectively. This requires proper orientation of the bracket in the hand of the orthodontist, with the gingival side facing downward for lower teeth and facing upward for the upper teeth.

Historically, an orthodontist stocks a plurality of brackets for each tooth, with the brackets often supplied loose in boxes. Each bracket was retrieved from a respective one of the boxes and oriented for application to the patient's tooth. When the orthodontist undertakes to retrieve the brackets from the boxes at chair-side, considerable professional time is consumed, while care must be taken to insure that the correct bracket is retrieved for a given tooth and is oriented correctly to have its pad coated with adhesive and applied to the tooth. The orthodontist will typically stock one type or model of appliance that is most commonly suitable for a patient, plus alternative types or sizes for use as the case may indicate.

The trend toward the use of a set-up tray has resulted in better use of the orthodontist's chair-side time in installing orthodontic appliances. This time has been replaced by technician or assistant time in loading the set-up trays employing many of the same motions and careful attention previously employed by the orthodontist. Set-up trays are usually hand-size trays or cards having compartments or sticky adhesive-coated pad areas to hold the individual appliances in a predetermined arrangement for pick-up by the orthodontist at tray side. Set-up trays of the type that not only hold the brackets for the orthodontist but present tooth-specific, single-doses of adhesive for use with each respective bracket are described in U.S. Pat. Nos. 6,213,767 and 6,482,003. Simpler set-up trays that hold only the brackets are also common.

Whether done by the practitioner or the practitioner's assistant, the efficiency of dental practice and the reduction of error occurrence can be improved by better ways for supplying and handling dental supplies.

### SUMMARY OF THE INVENTION

One aspect of the invention is an orthodontic appliance organizer. The orthodontic appliance organizer includes a storage component for storing an orthodontic appliance. The orthodontic appliance organizer includes an organizer tray that has a first panel and a hinged second panel coupled to the first panel. The hinged second panel includes a plurality of appliance holders thereon, each appliance holder having structure for holding an orthodontic appliance. In addition, the hinged second panel rotates relative to the first panel for providing access to the storage component of the orthodontic appliance organizer.

Another aspect of the invention is an orthodontic appliance organizer. The orthodontic appliance organizer includes a set-up tray support member for supporting a set-up tray. The orthodontic appliance organizer also includes an organizer tray having support for the set-up tray support member thereon. The organizer tray includes a plurality of appliance holders having structure for holding the orthodontic appliances. The orthodontic appliance organizer also includes a storage component having a top for supporting the organizer tray. The storage component also includes a side extending transversely relative to the top including compartments for storing the orthodontic appliances and allowing the user to access and transfer the orthodontic appliances between the storage component and the organizer tray.

In another aspect of the invention is an orthodontic appliance organizer. The orthodontic appliance organizer includes an organizer tray defining an aperture therein and including a plurality of appliance holders. The appliance holders have structure to hold the orthodontic appliances. The orthodontic appliance organizer also includes a storage component supporting the organizer tray. The storage component defines a recess that can be accessed by a user through the aperture in the organizer tray. The storage component also has storage compartments corresponding to appliance holders in the organizer tray. The combination of the organizer tray and the storage component when supporting the organizer tray aligns the aperture and the recess to define a bin.

### BRIEF DESCRIPTION OF THE DRAWINGS

FIG. 1 is a perspective view of one version of an orthodontic set-up tray of the prior art.

FIG. 2 is a perspective view of one embodiment of an orthodontic bracket package and carrier according to certain principles of the present invention.

FIG. 2A is an enlarged view of a portion of the package and carrier of FIG. 2.

FIG. 3 is a perspective view of an orthodontic bracket organizer according to certain embodiments of the present invention, in a closed condition.

FIG. 3A is a disassembled perspective view of the upper sliding portion of the organizer of FIG. 3.

FIG. 3B is a disassembled perspective view of the lower base portion of the organizer of FIG. 3.

FIG. 4 is a cross-sectional elevational view of the organizer tray of FIG. 3 taken along line 4-4 of FIG. 3.

FIG. 5 is a perspective view, similar to FIG. 3, of an organizer in an open condition.

FIG. 6 is a perspective view of the organizer of FIG. 3, showing the organizer in use.

FIG. 6A is an enlarged perspective view of a portion of FIG. 5.

FIG. 7 illustrates an exploded view of an alternate embodiment of an orthodontic appliance organizer.

FIG. 8 illustrates a perspective view of the orthodontic appliance organizer of FIG. 7 having one panel rotated to provide access to storage compartments in the storage component.

FIG. 9 illustrates a perspective view of a fully stocked orthodontic appliance organizer of FIG. 7.

FIG. 10A illustrates a perspective view of multiple stacked orthodontic appliance organizers of FIG. 7.

FIG. 10B illustrates a perspective view of multiple stacked set-up tray support members of the orthodontic appliance organizer of FIG. 7.

FIG. 10C illustrates a perspective view of multiple stacked organizer trays taken from an orthodontic appliance organizer of FIG. 7.

FIG. 10D illustrates a perspective view of multiple stacked storage components taken from an orthodontic appliance organizer of FIG. 7.

FIG. 11 illustrates a cross sectional view of multiple stacked orthodontic appliance organizers taken through line 11-11 of FIG. 10A.

FIG. 12 illustrates a side elevational view of multiple stacked storage components taken from an orthodontic appliance organizer of FIG. 7.

### DETAILED DESCRIPTION

In most dental practices, a set-up tray of some sort is prepared by a dental assistant and placed at chair-side next to a patient before the dental practitioner enters the treatment area for a dental appointment. This is particularly true in orthodontic practices for appointments to install orthodontic appliances on patients. In such orthodontic practices, an orthodontic set-up tray is typically loaded with orthodontic appliances that have been pre-selected by the practitioner for treatment of a patient for whom an appointment has been scheduled to install the appliance. One of several such prior art set-up trays that are in use by orthodontists is illustrated in FIG. 1.

In FIG. 1 is illustrated a set-up tray 10 of the type described in U.S. Pat. No. 6,482,003, hereby expressly incorporated by reference herein, and which is used in the description of the invention below. It is one prior art example of several types of set-up trays that can be used with the present invention. The particular set-up tray 10 that is illustrated and described is, in addition to being a set-up tray, a delivery system for delivering orthodontic adhesive in individual bracket doses for securing orthodontic brackets to teeth, although the adhesive delivery feature is not a necessary feature of any set-up tray for use with the present invention. The set-up tray 10 includes a resilient foam-board base 11 having a smooth plastic surface 12 that contains bracket mounting areas 13 and 14, lying on opposite sides of a centerline 19, that are coated with tacky pressure-sensitive adhesive. The area 13, when held by an orthodontist, will lie along the side of the tray 10 that faces a patient and will hold brackets 30 for the patient's upper dental arch, while the area 14 will lie along the side toward the orthodontist and will hold brackets for the patient's lower arch. The brackets 30 will be arranged on the tray in the order of the teeth in the patient's mouth, with staging areas 15 on the left side of the tray containing, from left to right, brackets for the right second molar through the right central incisor, and staging areas 16 on the right side of the tray containing, from left to right, brackets for the left central incisor through the right second molar. In the center of the tray 10 are provided enclosures 17 containing single doses of adhesive corresponding to each of the brackets, and cups 18 for holding primers or cleaning liquid.

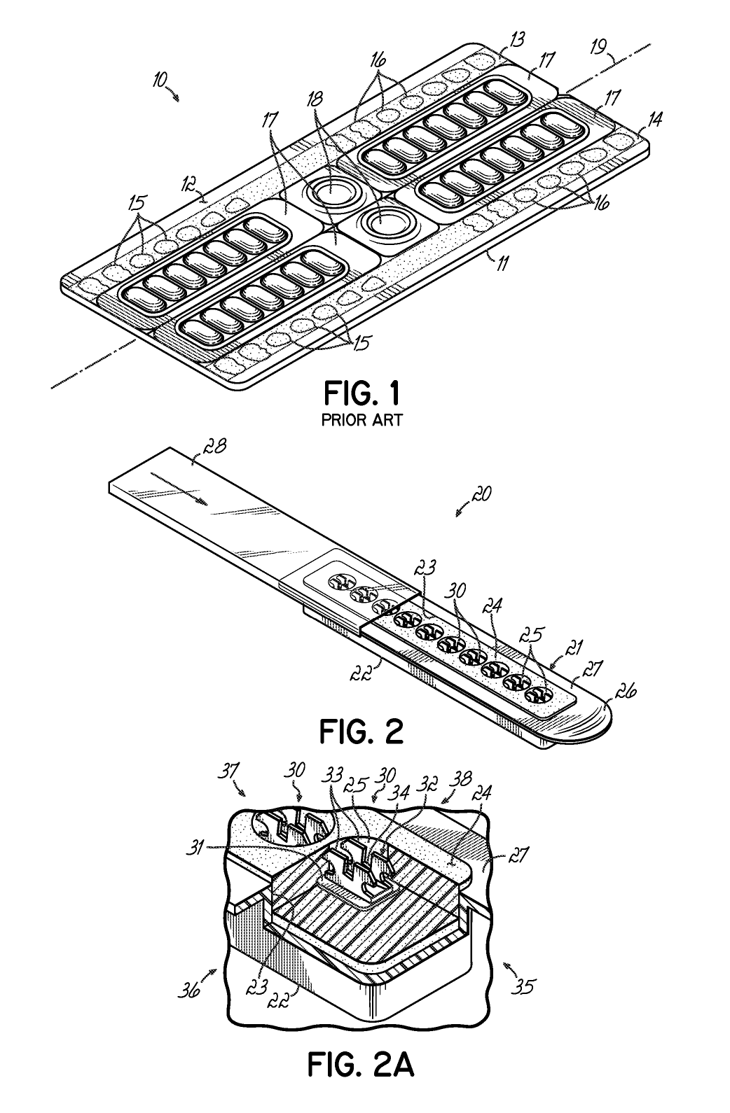

FIG. 2 illustrates a package 20 embodying certain features of the present invention. The package 20 includes an elongated carrier 21 having a plastic base 22 with an elongated recess 23 formed therein. The cavity contains a flexible foam insert 24 having a plurality of cavities 25 therein adapted to receive and to snugly but releasably hold an orthodontic bracket 30, as illustrated in more detail in FIG. 2A. The base 22 of the carrier 21 has a tab or handle 26 formed at one end thereof that is part of a flat flange 27 that extends in a plane around the recess 23. The package 20 includes a clear plastic cover 28 that slides over the carrier 21, having internal channels formed therein into which the flange 27 of the base 22 fit. The cover 28 covers the brackets 30 and keeps them in the cavities 25 for shipment from the bracket manufacturer.

The brackets 30 have pads or bases 31 to which is attached or integrally formed a bracket body 32 that is typically composed of a pair of tie wings 33 in which is formed a generally horizontal archwire slot 34. As illustrated in FIGS. 2 and 2A, the brackets 30 are packaged in the cavities 25 of the carrier 21 with base 32 facing down so that the tie wings can be gripped easily with tweezers to remove the bracket from the package 20. Further, the brackets 30 may each be considered as having four sides 35-38 that include a gingival side 35 that will face the gum when the bracket 30 is mounted on a patient's tooth. The brackets 30 are inserted into the cavities of the package 20 oriented such that the gingival side 35 of the bracket 30 faces the handle or tab 26 of the carrier 21. The other sides 36-38 include, proceeding clockwise around the bracket 30, a first mesial/distal side 36, an occlusal side 37 that is opposite the gingival side 35, and a second mesial/distal side 38 that is opposite the first mesial/distal side 38. For upper right and lower left brackets, side 36 is the mesial side of the bracket and side 38 is the distal side of the bracket; and for upper left and lower right brackets, side 36 is the distal side of the bracket and side 38 is the mesial side of the bracket.

The packages 20 have a plurality of the cavities 25 in a line. For brackets of more often used prescriptions, the number of cavities 25 in the plurality is typically seven to ten. The packages 20 may be made with other numbers of cavities 25. Three to five brackets per package may be a practical number, particularly for brackets of less often used appliance prescriptions.

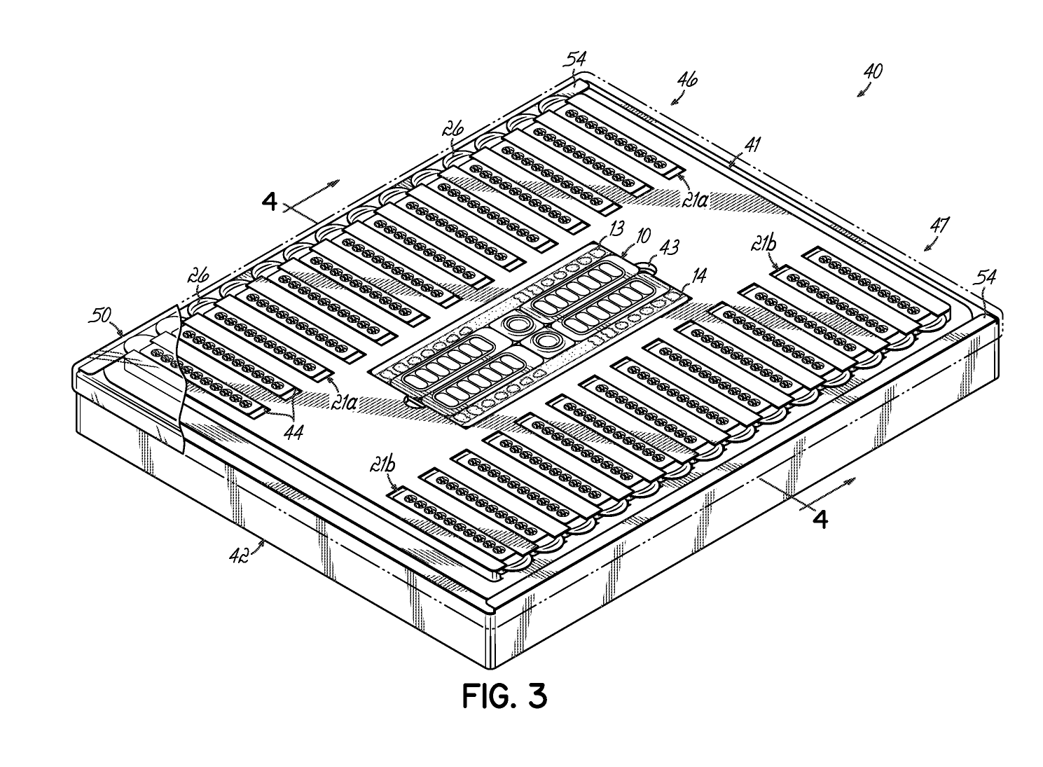

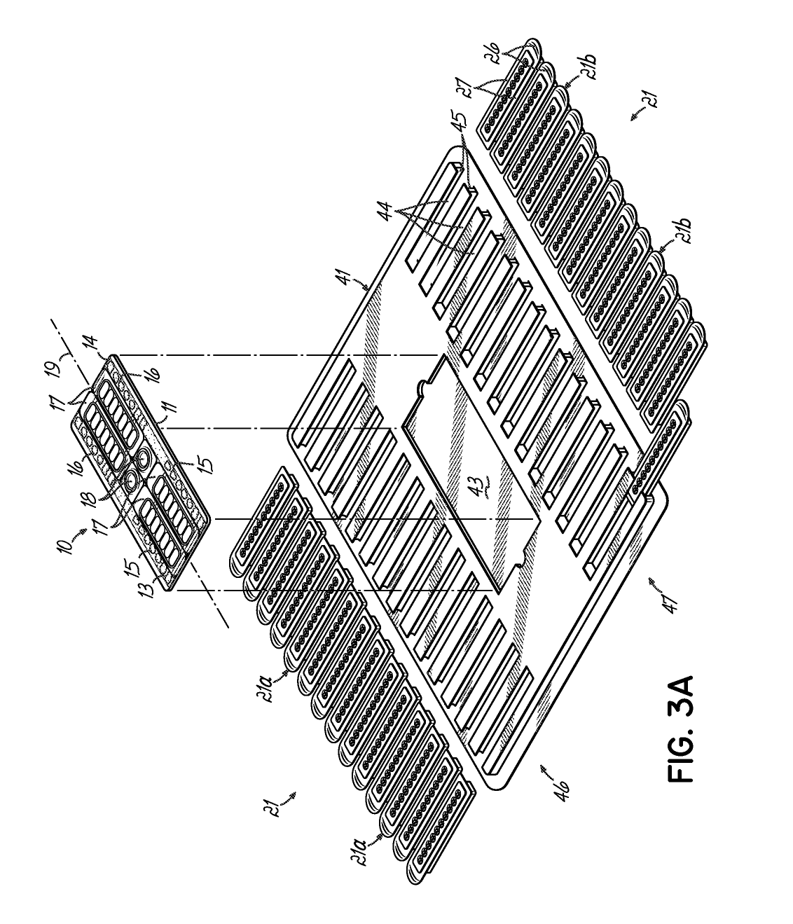

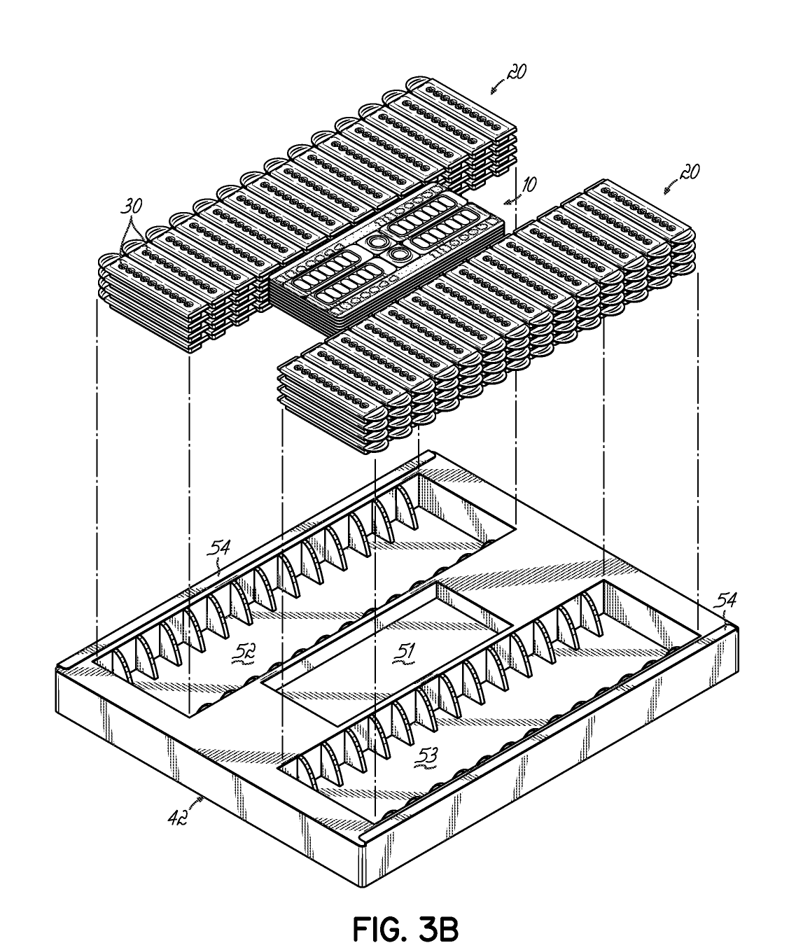

According to a method of the present invention, brackets 30 are loaded onto a set-up tray 10 from the carriers 20 by personnel at a treating orthodontic practitioner's office with the use of an organizer 40, illustrated in FIG. 3, also according to the present invention. The organizer 40 includes an organizer tray 41 and a storage base 42. Both the organizer tray 41 and storage base 42 is each preferably made of a respective integral piece of molded plastic, although metal or other materials may be used.

The organizer tray 41 has a four-sided, set-up-tray-receiving recess 43 formed at its center and a plurality of appliance holders 44. The holders are in the form of three-sided, open-ended, carrier-receiving recesses or slots 44, half of which are formed along one side of the organizer tray 41 and half formed along the opposite side of the organizer tray 41. The holders 44 are configured to hold the appliances 30, preferably in packages of appliances, such as the carriers 21. The number of the carrier-receiving recesses 44 is equal to the maximum number of brackets 30 that will form an orthodontic brace, typically twenty-eight.

As illustrated in FIGS. 3 and 3A, the organizer tray 41 is prepared by placing in the recess 43 an empty set-up tray 10 that is to be loaded with brackets from carriers 21. Carriers 21 containing brackets 30 are inserted into the slots 44 of the organizer tray 41, with their covers 28 removed, by sliding the flanges 27 onto grooves 45 in the sides of the slots 44, with the handles or tabs 26 of the carriers 21 at the open ends of the carrier-receiving recesses 44. Each slot 44 receives a carrier 21 containing brackets for a different one of the teeth. The carriers 21 containing the different brackets 30 of a set are loaded into the slots 44 of the organizer tray 41 in the same relative order as the staging areas 15 and 16 are arranged on the set-up card, with the upper bracket carriers 21, carriers 21a, along the side 46 that is adjacent the upper bracket mounting area 13 of the set-up tray 10, while the carriers 21 and with the lower bracket carriers 21, carriers 21b, along the side 47 that is adjacent the lower bracket mounting area 14 of the set-up tray 10. That is, the fourteen slots 44 along the side 46 of the organizer tray 41 contain brackets 30 for the upper teeth, arranged, left to right with brackets for the upper right second molar to the upper left second molar, and the fourteen slots 44 along the side 47 of the organizer tray 41 contain brackets 30 for the lower teeth, arranged, left to right with brackets for the lower right second molar to the lower left second molar.

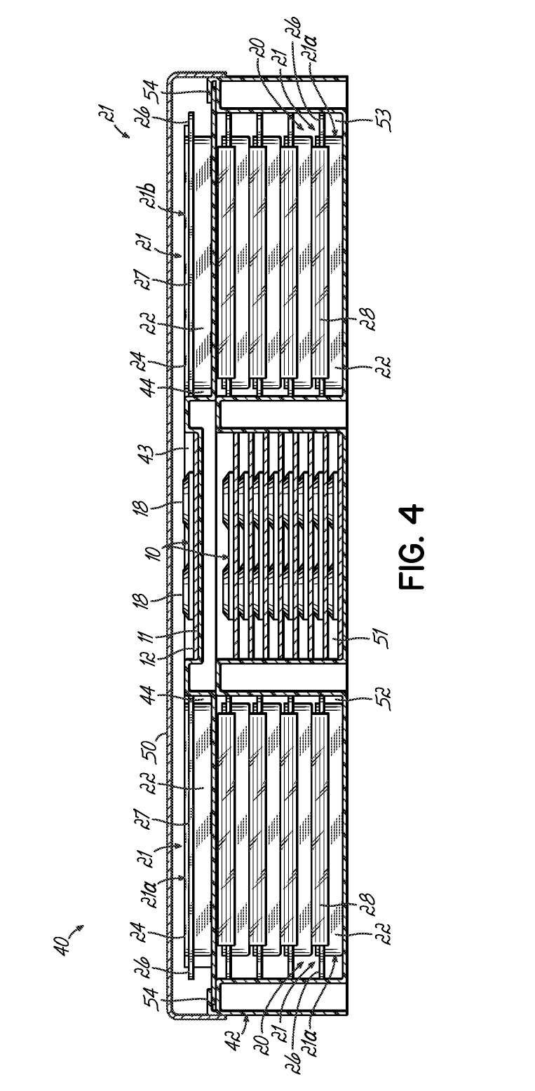

The base 42 contains hollow storage compartment 51-53, as illustrated in FIGS. 3B and 4. The compartment 51 is directly beneath the recess 43 in the organizer tray 41 when the tray 41 is in a closed position over the base 42 (FIG. 3). The compartment 51 contains a supply of empty set-up trays 10. Similarly, the compartment 52 is directly beneath the slots 44 on side 46 of the organizer tray 41 when the tray 41 is in this closed position and holds a supply of carriers 21a that contain upper brackets 30; and the compartment 53 is directly beneath the slots 44 on side 47 of the organizer tray 41 when in the closed position and holds a supply of carriers 21b that contain lower brackets 30.

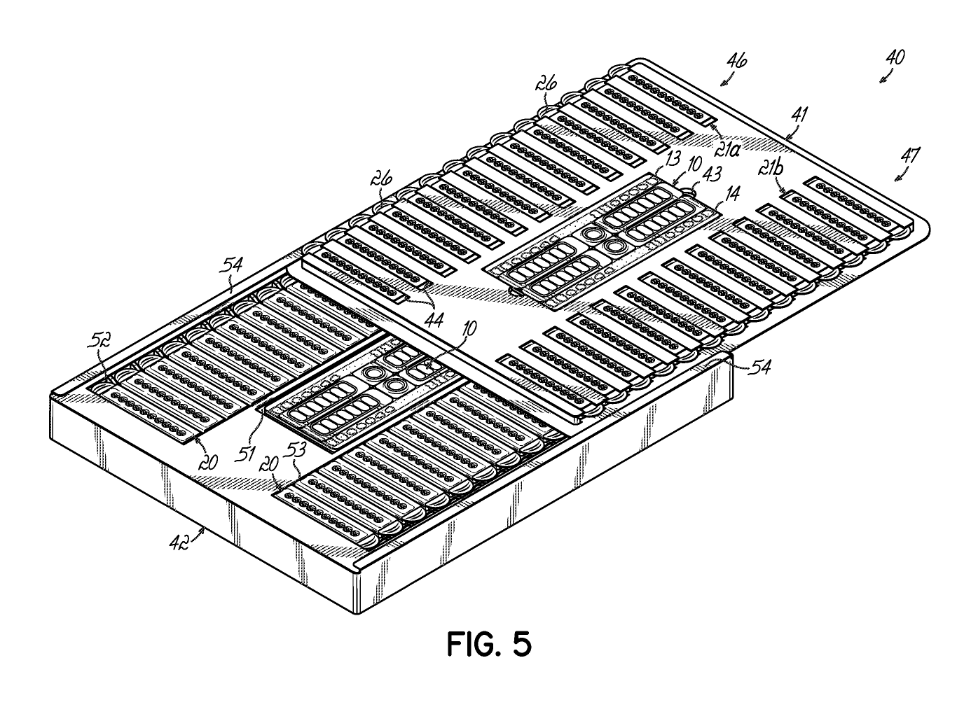

The organizer tray 41 forms a cover to the compartments 51-53 in the base 42, and slides over the base 42 with its outer edges along sides 46 and 47 fitting into grooves 54 along the sides of the base 42 adjacent compartments 52 and 53, respectively. The organizer tray 41 is shown in its closed position over the base 42 in FIG. 3 and in a partially open position in FIG. 5.

Further, a lid 50 may be provided to cover the organizer tray 41 so that the entire organizer 40 can be stored when partially empty carriers 21 are present in the slots 44 of the organizer tray 41. This allows the organizer 40 to be stored in any condition at the office of the orthodontic practitioner. Typically, a slotted cabinet or rack (not shown) is provided at the practitioner's office for storage of organizer trays 40 containing different bracket prescriptions or types.

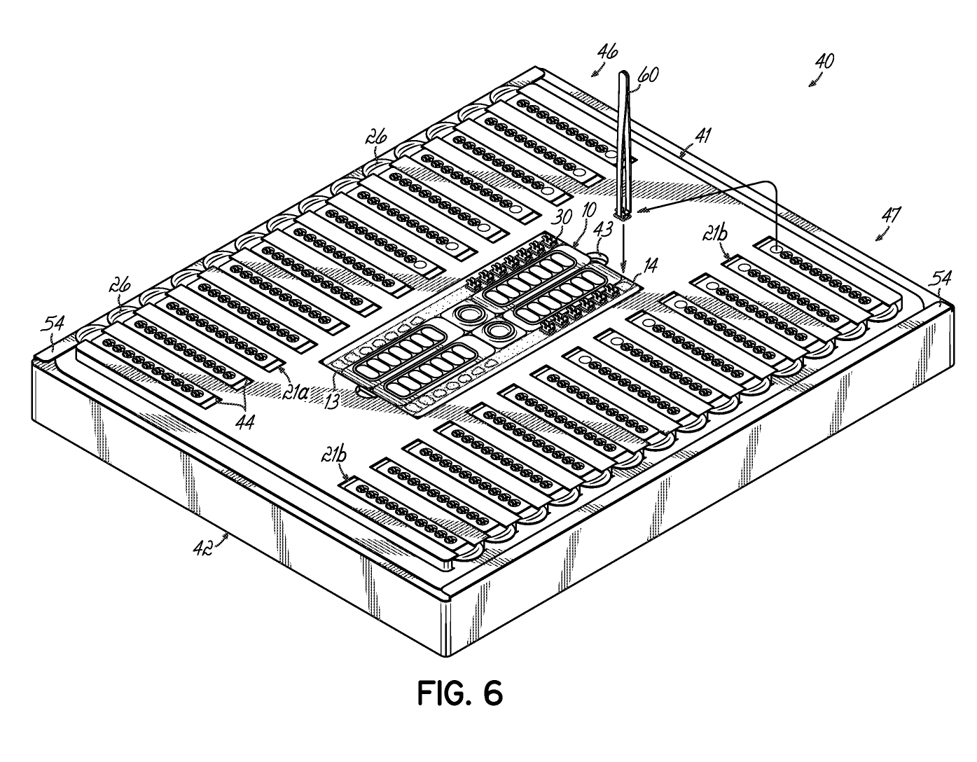

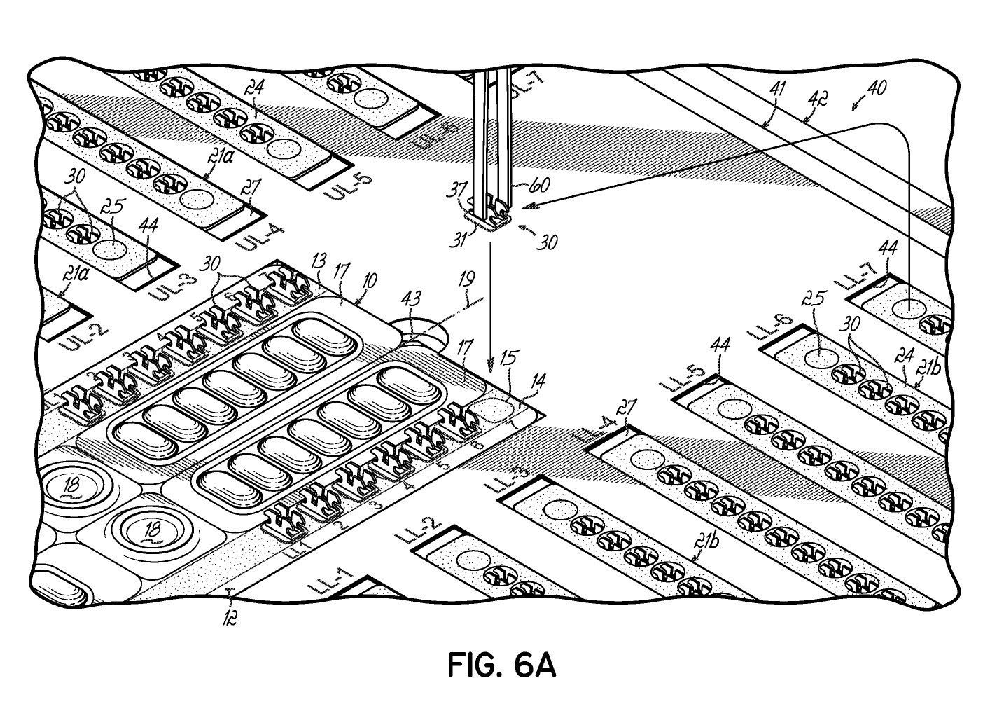

The use of the organizer 40 is illustrated in FIGS. 6 and 6A. The contemplated typical use of the organizer 40 involves the use of the organizer tray 41 by an orthodontic assistant to load a set-up tray 10 with one set of orthodontic brackets 30 to provide to the treating practitioner in installing the brackets 30 on the teeth of a patient. The assistant may use the organizer tray 41 separate from the base 42, or with the base 42 still attached. In loading a set-up tray 10, supported in the recess 43 at the center of the organizer tray 41, the assistant may use an instrument such as a pair of tweezers 60 to transfer each bracket 30 of a set from a hole 25 in a carrier 21 onto a staging area 15 for the particular bracket 30 on the set-up tray 10. The brackets 30 are placed on the staging areas 15 of the set-up tray 10 with their occlusal sides 37 facing the centerline 19 of the set-up tray 10. Because the brackets 30 are provided in the carriers 21 with their gingival edges facing the handles 26 of the carriers 21, and the carriers 21 are loaded into the slots 44 on the organizer tray 41, the brackets 30 are oriented on the organizer tray 41 so their occlusal edges face the centerline 19 of the set-up tray 10 that is supported in the recess 43. Accordingly, a mere translation, with no rotation, is all that is required to load a bracket 30 from a carrier 21 to the set-up card 19.

The organizer tray 41 is, in the preferred embodiment, loaded with supplies of brackets 30 from the compartments 52 and 53 beneath the corresponding slots 44 of the tray 41. This loading is carried out by removing one of the packages or carriers 21 from the compartment 52 or 53 and sliding the flanges 27 of the carriers 21 into the slots 45 of the tray 41, with the handles 26 of the carriers 21 facing away from the recess 43 that holds the set-up tray 10. In an alternative embodiment, the carriers 21 and the holders 44 may be configured so that the appliances 30 can be transferred from the alternatively configured versions of the packages 21 to alternatively configured versions of the holders 44, with their orientations preferably preserved in the transfer.

When a set-up 10 tray is loaded as described above, an orthodontist may hold the set-up tray 10 with the centerline horizontal and the side holding the bracket mounting areas 13 facing the patient, and then similarly transfer the brackets 30 with tweezers onto the teeth of the patient. Such transfer requires only rotating the pad 31 of the bracket 30 from a horizontal to a vertical plane. Further reorientation of a bracket 30 is not necessary to place the bracket 30 in its correct orientation for mounting on the patients tooth

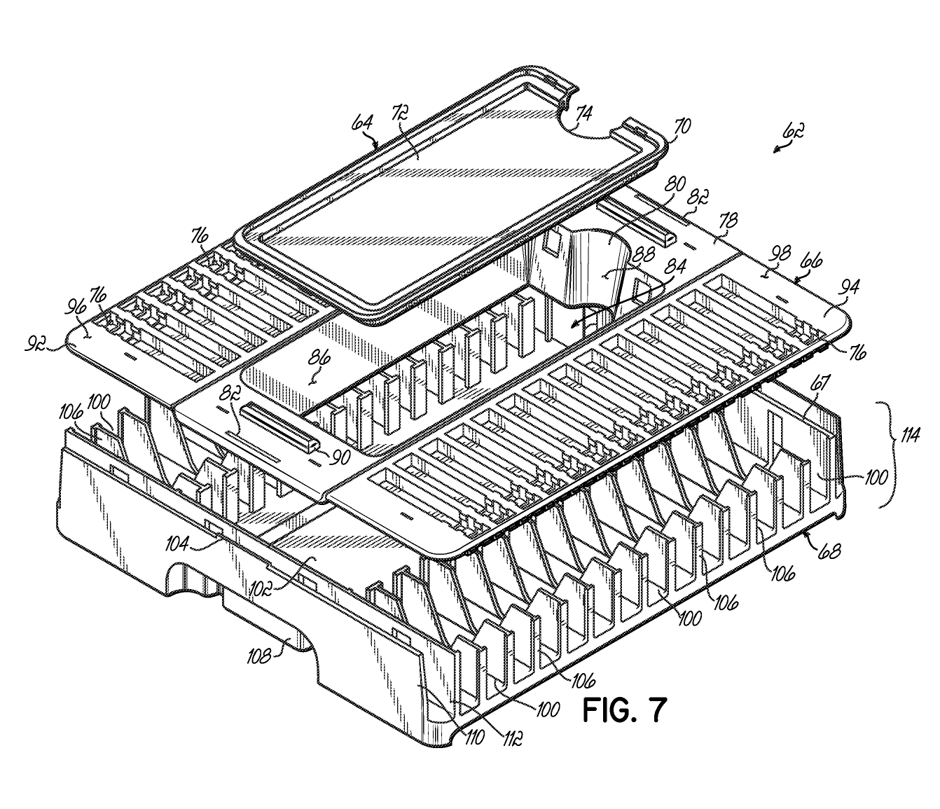

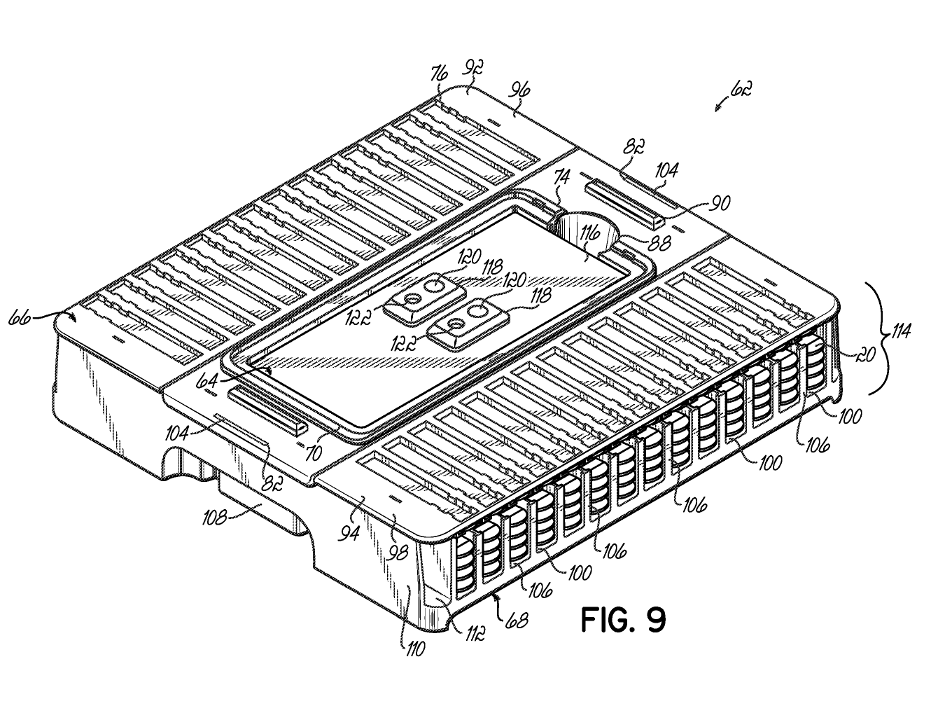

Referring now to FIG. 7, an alternative embodiment of an orthodontic appliance organizer 62 is provided with advantageous features described below. The orthodontic appliance organizer 62 includes three main components, a set-up tray support member 64 for supporting a set-up tray, such as set-up tray 10 of FIG. 1 or the set-up tray described in FIG. 9 hereinbelow, an organizer tray 66 for organizing a plurality of orthodontic appliances to be used with a patient, and a storage component 68 for storing the orthodontic appliances. In the illustrated embodiment, the storage component 68 acts as a base and supports the other two components. Other embodiments have the storage component 68 as an intermediary between the base and the organizer tray 66 or other configuration. The components are generally formed out of a plastic material, such as a high impact plastic. In addition, the organizer tray 66 is commonly formed of polypropylene or polyethylene, however, other embodiments use other materials.

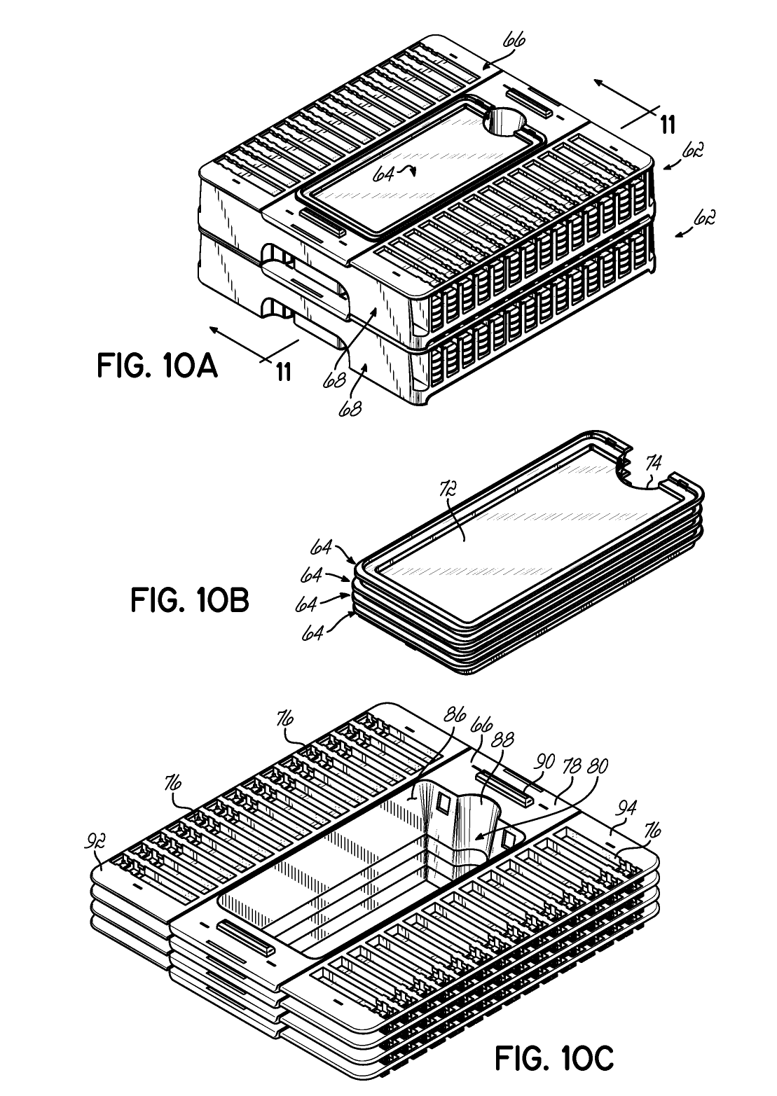

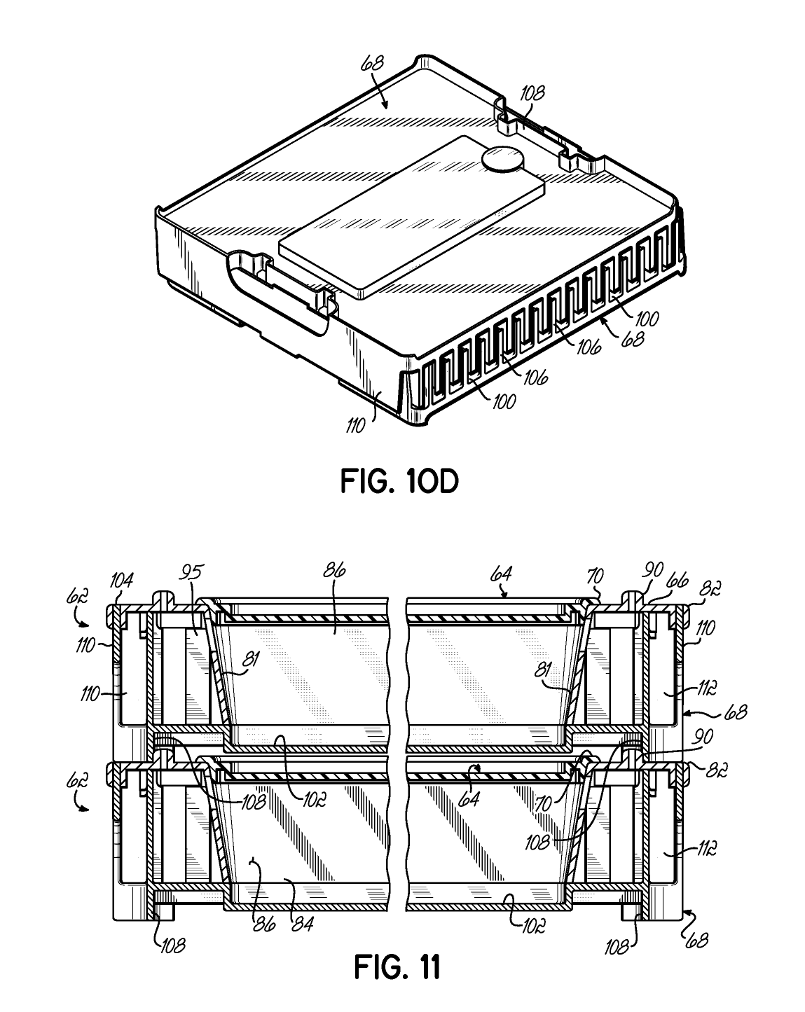

The set-up tray support member 64 holds a set up tray for preparing the orthodontic appliances for application to the teeth of the patient. The set up tray can hold adhesive, tools, and help keep the orthodontic appliances positioned for application. The set-up tray support member 64 of the illustrated embodiment is formed of a plastic or rubber material, however, other embodiments use other types of materials. The set-up tray support member 64 includes a rim 70. The rim 70 interacts with the organizer tray 66 to support the set-up tray support member 64 when the set-up tray support member 64 is placed upon the organizer tray 66. The set-up tray support member 64 defines a recessed area 72 for supporting a set-up tray (not shown). The recessed area 72 also cooperates with the rim 70 for providing sufficient structure for maintaining the original orientation of the set-up tray support member 64 when placed upon the organizer tray 66. In addition, the structure enables the stacking of multiple set-up tray support members 64 as described in further detail hereinbelow with respect to FIG. 10B. The set-up tray support member 64 also defines an aperture 74 along the rim 70 of the set-up tray support member 64. The aperture 74 of the illustrated embodiment is located along one of the shorter sides of the set-up tray support member 64. Other embodiments have the aperture 74 placed in different locations along the rim 70 or even within the interior of the set-up tray support member 64. The aperture 74 allows the orthodontist or other dental professional to easily grab and remove the set-up tray support member 64 from the organizer tray 66. In the illustrated embodiment, the aperture 74 is a semicircle configuration, however, other embodiments have other types of configurations.

The organizer tray 66 is designed with a plurality of appliance holders 76 formed therein designed to receive the carriers 21. In the illustrated embodiment, the appliance holders 76 are arranged on opposite sides of the organizer tray 66. The organizer tray 66 is divided into three sections or panels. The first panel 78 is the central panel that includes an aperture 80 formed therein. The set-up tray support member 64 covers at least a portion of this aperture 80 when placed upon the organizer tray 66. The first panel 78 does not define any appliance holders 76. The first panel 78 also includes slots 82 for facilitating the combination of the organizer tray 66 with the storage component 68. Moreover, the combination of the first panel 78 and the storage component 68 forms a bin 84 having side walls 86 and a curved recess 88. The curved recess 88 corresponds to the aperture 74 defined in the set-up tray support member 64. Thus, the dental professional can easily place their fingers through the aperture 74 and into the curved recess 88 for lifting the set-up tray support member 64 off of the organizer tray 66. The bin 84 is generally used to store set-up trays in the illustrated embodiment, however, other items can be stored in the bin 84 in other embodiments. The first panel 78 also includes plateaus 90 that are designed to cooperate with areas on the storage component 68 to facilitate stacking of multiple ones of the orthodontic appliance organizer 62.

The organizer tray 66 also includes a second panel 92 and a third panel 94 defining a plurality of appliance holders 76 defined therein. The appliance holders 76 are open along the top surface 96 of the second panel 92 and the top surface 98 of the third panel 94 to enable visual inspection by the dental professional. In addition, the appliance holders 76 are designed to enable the carriers 21, or other similar device, to be slid in and out with ease. There are fourteen appliance holders 76 on each one of the panels 92, 94 of the organizer tray 66 corresponding to the fourteen teeth on the upper arch and the lower arch. Accordingly, the organizer tray 66 positions the carriers 21, or other device, so that the orthodontic appliances for a specific tooth are in an appliance holder 76 for that spot. The second and third panels 92, 94 are hinged around axes defined by the intersection of the first panel 78 with the second and third panels 92, 94. The second panel 92 rotates clockwise from the position illustrated in FIG. 7 to provide access to the storage component 68. Similarly, the third panel 94 can rotate counter-clockwise from the position illustrated in FIG. 7 for providing access to the storage component 68.

The storage component 68 includes a plurality of storage compartments 100 used to hold a plurality of packages 20 or other similar assemblies. Each one of the storage compartments 100 corresponds to an appliance holder 76 on the organizer tray 66. The storage component 68 also includes a recess 102 designed for providing space for formation of the bin 84 by combining the first panel 78 of the organizer tray 66 and the storage component 68. The storage component 68 includes ridges 104 that cooperate with the slots 82 of the organizer tray 66 for rigidly combining the organizer tray 66 to the storage component 68. The storage component 68 also includes shelves 106 that help define the storage compartments 100 that are appropriately spaced to hold the packages 20 or similar assemblies. The storage component 68 also includes flanges 108 upon the bottom that are useful in stacking multiple ones of the orthodontic appliance organizer 62 described in more detail hereinbelow with respect to FIG. 11. The storage component 68 also includes grips 110 and grip areas 112 that are configured to permit the dental professional to easily grasp and move the orthodontic appliance organizer 62 to different locations.

In use, the orthodontic appliance organizer 62 enables quick and easy application of the orthodontic appliances to a patient's mouth. Moreover, the design simplifies the process for the dental professional to restock and arrange their orthodontic appliance inventory. Initially, the storage component 68 is filled with a plurality of packages 20 containing the orthodontic appliances. In the illustrated embodiment, the storage component 68 is designed to be able to hold four packages 20 stacked one upon each other. Those skilled in the art recognize that other embodiments can hold a different number of packages 20 in the storage component 68. Furthermore, other types of orthodontic appliance packages 20 are used besides those described in FIG. 2. The filling of the storage component 68 with packages 20 can be before the entire product is shipped to the dental professional or can be filled by the dental professional or others after manufacture and assembly.

After the storage component 68 is filled with packages 20 or similar assemblies, the organizer tray 66 is placed onto the top 67 of the storage component 68 by aligning the aperture 80 with the open space 102. The bin 82 is formed by the combination of the first panel 78 of the organizer tray 66 and the storage component 68. The bin 82 is sized for filling with set-up trays. Other embodiments fill the bin 82 with other materials and fill the recess 102 or the bin 82 at other times. The slots 82 and the ridges 104 cooperate securely attaching the organizer tray 66 to the storage component 68. The set-up tray support member 64 is then placed over the aperture 80 of the first panel 78 of the organizer tray 66 concealing the set-up trays. The rim 70 of the set-up tray support member 64 is larger than the aperture 80 and therefore provides structure for supporting the set-up tray support members 64. The orthodontic appliance organizer 62 is then ready for use by a dental professional.

The dental professional places the orthodontic appliance tray 62 in a location near the patient. The dental professional removes one of the carriers 21 from the packages 20 located in the storage component 68. The storage component 68 enables lateral access of the carriers 21 because the storage compartments 100 are open around the sides 114 extending transversely to the storage component 68. The removed carrier 21 is slid into the appliance holder 76 positioned directly above the dedicated one of the storage compartments 100 that the carrier 21 was removed from. Each carrier 21 corresponds to an individual tooth that will be handled by the dedicated appliance holder 76 placed directly above the dedicated one of the storage compartments 100. Therefore, keeping the orthodontic appliances organized by tooth is very simple because of the design of the storage component 68 and organizer tray 66. In addition, the dental professional can easily move and organize their inventory based on the design. As in the embodiments illustrated in FIG. 3 and the like, there is a color coding scheme used with the carriers 21 or the appliance holders 76 indicating to the dental professional the appliances that correspond to certain teeth.

The dental professional can then pull out the set-up tray support member 64 using the aperture 74. The dental professional grabs one of the set-up trays stored inside of the bin 84. The set-up tray support member 62 is placed right back over top of the aperture 80 and the set-up tray is placed inside of the recessed area 72 of the set-up tray support member 64. The dental professional can then remove orthodontic appliances and transfer them to the set-up tray for preparation and application to a dedicated tooth.

Each carrier 21 of the illustrated embodiment holds ten brackets for use on the same tooth. In addition, the illustrated embodiment has a storage component 68 that holds four packages 20 each containing a carrier 21. Therefore, the orthodontic appliance organizer 62 of the illustrated embodiment holds enough orthodontic appliances for forty patients. Replacement and restocking the orthodontic appliance inventory is a simple procedure. Once the orthodontist or other dental professional has gone through forty patients, the second panel 92 and third panel 94 are rotated for providing access to the storage compartments 100 of the storage component 68. The plastic covers 28 remain inside of the storage compartments 100 and can be removed or reused. New sets of packages 20 or carriers 21 are placed into the storage compartments 100 of the storage component 68 enabling additional patients to be serviced by the orthodontic appliances contained therein.

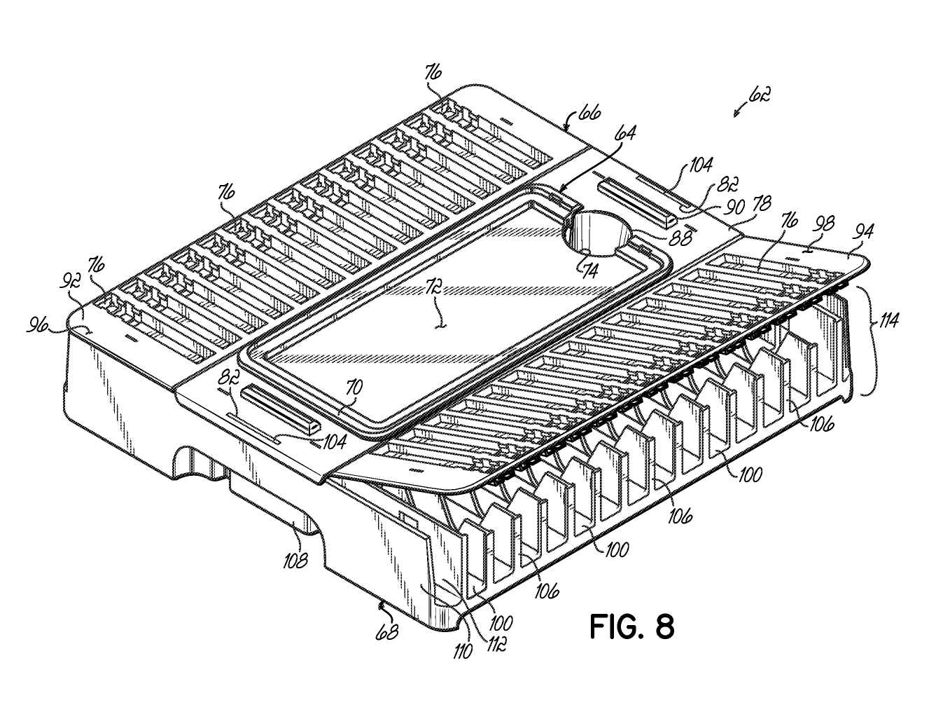

Referring now to FIG. 8, an assembled view of the orthodontic appliance organizer 62 is illustrated. This view illustrates that the set-up tray support member 64 covers most of the aperture 80 except for the area defined by the aperture 74 and the curved recess 88. In addition, the slots 82 are filled up with the ridges 104 for securing the organizer tray 66 to the storage component 68. FIG. 8 also illustrates the third panel 94 rotated counter-clockwise facilitating access to the storage compartments 100 of the storage component 68. Accordingly, the dental professional can rotate the second panel 92 and the third panel 94 to stock the packages 20 in the storage compartments 100.

Referring now to FIG. 9, the assembled orthodontic appliance organizer 62 is completely stocked. A total of four packages 20 are stored in each one of the storage compartments 100. These packages 20 each contain a carrier 21 that can be slid out through the sides 114 of the storage component 68 and inserted into the appliance holder 76 of the organizer tray 66. In addition a set-up tray 116 has been placed into the recessed area 72 of the set-up tray support member 64. The set-up tray 116 has a different design than the set-up tray 10 illustrated in FIG. 1. The set-up tray 116 includes a couple of platforms 118 in the center of the set-up tray 116. The platforms 118 each contain a pad 120 and a recess 122. The pad 120, in some embodiments, is used as a location for the dental professional to place the orthodontic appliance that is to be applied to the teeth prior to application. The recess 122 is an area to hold the adhesive or other substance in order to fix the appliance to the tooth.

Referring now to FIGS. 10A-D, the stacking of the different components and the complete orthodontic appliance organizer 62 is illustrated. FIG. 10A illustrates that an orthodontic appliance organizer 62 can be stacked upon another orthodontic appliance organizer 62. This is useful for the manufacturer of the device to be able to easily store these orthodontic appliance trays 62 for sale. FIG. 10B illustrates multiple ones of the set-up tray support member 64 stacked upon each other. The recessed area 72 of each set-up tray support member 64 fits into the corresponding recessed area 72 of the other set-up tray support member 64. This is similar to the stacking arrangement for trays used in restaurants and the like. Referring to FIG. 10C, multiples ones of the organizer tray 66 are stacked upon each other. The side walls 86 of each organizer tray 66 fits into the aperture 80 of another organizer tray 66 and the side walls 86 friction fit against the other corresponding side walls 86. FIG. 10D illustrates the storage component 68 stacked upon another storage component 68. Each storage component 68 has each of the storage compartments 100 receive one of the other shelves 106 of the storage component 68. The combination fits into a space almost the same size as one storage component 68 greatly improving the ability to store and stack multiples ones of the storage component 68.

Referring now to FIG. 11, a cross-section of FIG. 10A taken through line 11-11 illustrates the structure that enables multiples ones of the orthodontic appliance organizer 62 to be stacked upon each other. The illustrated embodiment is only one type of structure for facilitating stacking multiples ones of the orthodontic appliance organizer 62 and other embodiments use other structural configurations to facilitate stacking. The flanges 108 of the storage component 68 are designed to fit around the plateaus 90 of the organizer tray 66. The flanges 108 prevent the orthodontic appliance organizer 62 from being dislodged in the direction opposite to the storage compartments 100. In addition, the design also facilitates sliding of the orthodontic appliance tray 62 in the direction of the storage compartments 100 enabling ease of stacking and removal.

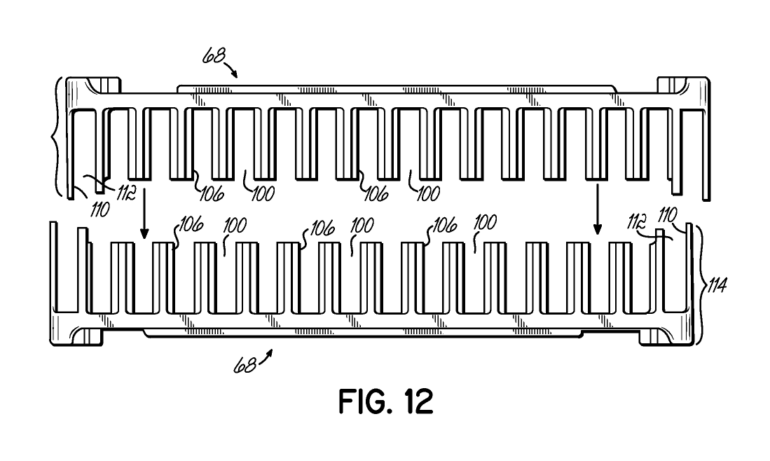

FIG. 12 illustrates the structure for combining multiples ones of the storage component 68 together. The shelves 106 are positioned for receipt by the storage compartments 100. In addition, on the sides 114 of the storage component 68 the grips 110 and the grip areas 112 are complementarily positioned. The grips 110 of one side 114 of the storage component 68 fit into the grip area 112 of another side 114 of a different storage component 68 compacting them both into an area close to the same volume as only one storage component 68. Therefore, all the different components of the orthodontic appliance organizer 62 can be stacked individually and the orthodontic appliance organizer 62 themselves can also be stacked upon one another.

The invention has been described in the context of exemplary embodiments. Those skilled in the art will appreciate that additions, deletions and modifications to the features described herein may be made without departing from the principles of the present invention.

Accordingly, the following is claimed:

1. An orthodontic appliance organizer comprising:
a storage component for storing an orthodontic appliance; and
an organizer tray including a first panel and a hinged second panel coupled to the first panel, the hinged second panel having a plurality of appliance holders thereon, each appliance holder having structure for holding the orthodontic appliance, wherein the hinged second panel rotates relative to the first panel for providing access to the storage component of the orthodontic appliance organizer.

2. The organizer of claim 1 further comprising:
a set-up tray support member with structure to restrain the orientation of a set-up tray relative to the organizer tray.

3. The organizer of claim 2 wherein:
the set-up tray support member is stackable upon a second set-up tray support member.

4. The organizer of claim 2 wherein:
the organizer tray includes support for the set-up tray support member and each appliance holder holds the orthodontic appliance in a restricted orientation for transfer to the set-up tray.

5. The organizer of claim 4 further comprising:
a set-up tray to be received by the set-up tray support member.

6. The organizer of claim 1 wherein:
the structure of the appliance holders for holding an orthodontic appliance includes structure configured to receive a package of appliances and to hold the package in a restricted orientation relative to the organizer tray.

7. The organizer of claim 1 wherein:
the structure of the appliance holders for holding the orthodontic appliance includes a recess having a pair of opposed slots on opposite sides thereof configured to receive a flange of a package of appliances and to hold the package in a restricted orientation relative to the organizer tray.

8. The organizer of claim 1 further comprising:
a hinged third panel coupled to the first panel, the hinged third panel having a plurality of appliance holders thereon, each appliance holder having structure for holding the orthodontic appliance, wherein the hinged third panel rotates relative to the first panel for providing access to the storage component of the orthodontic appliance organizer.

9. The organizer of claim 8 wherein:
the plurality of appliance holders, one for each appliance to be mounted on a plurality of upper teeth of a patient, arranged along the second hinged panel of the organizer tray, and one for each appliance to be mounted on a plurality of lower teeth of a patient, arranged along the third hinged panel of the organizer tray.

10. The organizer of claim 1 further comprising:
the storage component having a top for supporting the organizer tray and a side extending transversely relative to the top, the storage component having a plurality of storage compartments therein, each corresponding to one of the holders of the hinged second panel and each allowing a user to access and transfer the orthodontic appliances between the storage component and the organizer tray.

11. The organizer of claim 10 wherein:
the storage component is stackable upon a second storage component.

12. The organizer of claim 10 wherein:
the combination of the organizer tray and the storage component defines a bin for storing set-up trays.

13. The organizer of claim 12 further comprising:
a set-up tray support member for covering the bin when placed on the organizer tray.

14. The organizer of claim 1 wherein:
the orthodontic appliance organizer is stackable upon a second orthodontic appliance organizer.

15. The organizer of claim 1 wherein:
the organizer tray is stackable upon a second organizer tray.

16. An orthodontic appliance organizer comprising:
a set-up tray support member for supporting a set-up tray;
an organizer tray having support for the set-up tray support member thereon, the organizer tray including a plurality of appliance holders thereon, the appliance holders having structure for holding the orthodontic appliances; and
a storage component having a top for supporting the organizer tray and a side extending transversely relative to the top, the side including compartments for storing the orthodontic appliances and allowing a user to access and transfer the orthodontic appliances between the storage component and the organizer tray.

17. The organizer of claim 16 wherein:
the set-up tray support member includes structure to restrain the orientation of the set-up tray relative to the organizer tray.

18. The organizer of claim 16 wherein:
the set-up tray support member is stackable upon a second set-up tray support member.

19. The organizer of claim 16 wherein:
each appliance holder holds the orthodontic appliance in a restricted orientation for transfer to the set-up tray.

20. The organizer of claim 16 wherein:
the combination of the organizer tray and the storage component defines a bin for storing set-up trays.

21. The organizer of claim 20 wherein:
the set-up tray support member covers the bin when placed on the organizer tray.

22. The organizer of claim 16 wherein:
the structure of the appliance holders for holding an orthodontic appliance includes structure configured to receive a package of appliances and to hold the package in a restricted orientation relative to the organizer tray.

23. The organizer of claim 16 wherein:
the structure of the appliance holders for holding an orthodontic appliance includes a recess having a pair of opposed slots on opposite sides thereof configured to receive a flange of a package of appliances and to hold the package in a restricted orientation relative to the organizer tray.

24. The organizer of claim 16 wherein the organizer tray comprises:
a first panel;
a hinged second panel coupled to the first panel, the hinged second panel having a portion of the plurality of appliance holders thereon, wherein the hinged second panel rotates relative to the first panel for providing access to the storage component; and
a hinged third panel coupled to the first panel, the hinged third panel having a portion of the plurality of appliance holders thereon, wherein the hinged third panel rotates relative to the first panel for providing access to the storage component.

25. The organizer of claim 24 wherein:
along the second hinged panel of the organizer tray for each of a plurality of upper teeth of a patient there is a dedicated one of the plurality of appliance holders arranged for mounting on each one of the upper teeth; and
along the third hinged panel of the organizer tray for each of a plurality of lower teeth of a patient there is dedicated one of the plurality of appliance holders arranged for mounting on each one of the lower teeth.

26. The organizer of claim 16 wherein:
the storage component is stackable upon a second storage component.

27. The organizer of claim 16 wherein:
the orthodontic appliances are a different one of the orthodontic brackets of a set.

28. The organizer of claim 16 wherein:
the orthodontic appliance organizer is stackable upon a second orthodontic appliance organizer.

29. The organizer of claim 16 wherein:
the organizer tray is stackable upon a second organizer tray.

30. An orthodontic appliance organizer comprising:
an organizer tray defining an aperture therein, the organizer tray including a plurality of appliance holders, the appliance holders having structure to hold the orthodontic appliances; and
a storage component supporting the organizer tray, the storage component having a recess, wherein the aperture allows a user to access the recess through the organizer tray, the storage component having storage compartments corresponding to the appliance holders in the organizer tray;
wherein the combination of the organizer tray and storage component when supporting the organizer tray aligns the aperture and the recess to define a bin.

31. The organizer of claim 30 further comprising:
a set-up tray support member for supporting a set-up tray.

32. The organizer of claim 31 wherein:
the organizer tray having structure to support the set-up tray support member.

33. The organizer of claim 31 wherein:
the set-up tray support member includes structure to restrain the orientation of the set-up tray relative to the organizer tray.

34. The organizer of claim 31 wherein:
the set-up tray support member is stackable upon a second set-up tray support member.

35. The organizer of claim 31 wherein:
the bin is constructed and arranged to contain set-up trays.

36. The organizer of claim 31 wherein:
the set-up tray support member covers the bin when placed onto the organizer tray.

37. The organizer of claim 30 wherein:
the structure of the appliance holders for holding an orthodontic appliance includes structure configured to receive a package of appliances and to hold the package in a restricted orientation relative to the organizer tray.

38. The organizer of claim 30 wherein:
the structure of the appliance holders for holding an orthodontic appliance includes a recess having a pair of opposed slots on opposite sides thereof configured to receive a flange of a package of appliances and to hold the package in a restricted orientation relative to the organizer tray.

39. The organizer of claim 30 wherein the organizer tray further comprises:
a first panel defining the aperture;
a hinged second panel coupled to the first panel, the hinged second panel having a portion of the plurality of appliance holders thereon, wherein the hinged second panel rotates relative to the first panel for providing access to the storage component; and
a hinged third panel coupled to the first panel, the hinged third panel having a portion of the plurality of appliance holders thereon, wherein the hinged third panel rotates relative to the first panel for providing access to the storage component.

40. The organizer of claim 39 wherein:
along the second hinged panel of the organizer tray for each of a plurality of upper teeth of a patient there is a dedicated one of the plurality of appliance holders arranged for mounting on each one of the upper teeth; and
along the third hinged panel of the organizer tray for each of a plurality of lower teeth of a patient there is dedicated one of the plurality of appliance holders arranged for mounting on each one of the lower teeth

41. The organizer of claim 30 wherein:
the storage component is stackable upon a second storage component.

42. The organizer of claim 30 wherein:
the orthodontic appliances are a different one of the orthodontic brackets of a set.

43. The organizer of claim 30 wherein:
the orthodontic appliance organizer is stackable upon a second orthodontic appliance organizer.

44. The organizer of claim 30 wherein:
the organizer tray is stackable upon a second organizer tray.
